(19) United States
(12) Patent Application Publication
(10) Pub. No.: US 2022/0333082 A1
(43) Pub. Date: Oct. 20, 2022

(54) METHODS FOR DERIVING AUTOLOGOUS AND HYPOIMMUNOGENIC HAIR FOLLICLE CONTAINING SHEETS IN VITRO

(71) Applicant: Helmuth Heinrich Kunz, South Beaconsfield (GB)

(72) Inventor: Helmuth Heinrich Kunz, South Beaconsfield (GB)

(21) Appl. No.: 17/641,250

(22) PCT Filed: Jul. 3, 2020
(86) PCT No.: PCT/EP2020/068813

§ 371 Date: Mar. 8, 2022

Related U.S. Application Data
(60) Provisional application No. 62/872,379, filed on Jul. 10, 2019.

(51) Int. Cl. C12N 5/0775; C12N 5/071

(52) U.S. Cl. CPC C12N 5/0666; C12N 5/0627; C12N 2500/32; C12N 2506/1376; C12N 2510/00

(57) ABSTRACT

The present disclosure relates to a bioengineering process to derive hair follicles in vitro from the in vitro disposition and differentiation of autologous pluripotent stem cells and dermal papilla stem cells. The present disclosure also relates to the in vitro bioengineering of hypoimmunogenic hair follicles from allogenic pluripotent stem cells and dermal papilla stem cells. The present disclosure also relates to bioengineering of autologous and allogenic hypoimmunogenic hair follicles and hair follicle containing sheets with asymmetric disposition of hair shafts. The present disclosure also relates to a bioengineering process to derive hair follicle containing sheets in vitro from a biodegradable supportive grid and said in vitro derived hair follicles. The present disclosure also relates to the controlled asymmetry of the hair shaft on said hair follicle containing sheets. The present disclosure also relates to the field of cosmetic materials and method for reconstructing hair follicle containing materials in vitro.

### TECHNICAL FIELD

The present disclosure relates to a bioengineering process to derive hair follicles in vitro from the in vitro disposition and differentiation of autologous pluripotent stem cells and dermal papilla stem cells. The present disclosure also relates to the in vitro bioengineering of hypoimmunogenic hair follicles from allogenic pluripotent stem cells and dermal papilla stem cells. The present disclosure also relates to bioengineering of autologous and allogenic hypoimmunogenic hair follicles and hair follicle containing sheets with asymmetric disposition of hair shafts. The present disclosure also relates to a bioengineering process to derive hair follicle containing sheets in vitro from a biodegradable supportive grid and said in vitro derived hair follicles. The present disclosure also relates to the controlled asymmetry of the hair shaft on said hair follicle containing sheets. The present disclosure also relates to the field of cosmetic materials and method for reconstructing hair follicle containing materials in vitro.

### DESCRIPTION

Bioengineering definition: Application of engineering and biological principles for purposefully defining cellular behaviour and or cellular disposition within an engineered synthetic tissue and or organ. Furthermore, the designed modification of genetic information to code altered cellular functions with beneficial impact on biologically derived materials and/or cellular behaviour. In addition, the use of engineering and biological principles to create novel tissue and/or organs and or biologically derived materials.

The use of vertebrata adult progenitors or adult stem cells has enabled the derivation of a variety of cell types within said adult progenitor tissue. In addition, vertebrata embryonic stem cells which count with a higher differentiation potential enable deriving all adult cell types in said vertebrata species, particularly adult stem cells and terminally differentiated cells. Furthermore, the reprogramming process from differentiated cells towards higher pluripotency states (primed or naive) provides an inexhaustible cell source.

The reprogramming process from differentiated cells towards cells of higher pluripotency state may be achieved through the exogenous synthetic expression of transcriptional factors and or epigenetic modifiers that reinforce the core transcriptional network of said pluripotent state. The simplicity of this process has placed reprogrammed stem cells as a convenient cell source for bioengineering and regeneration.

The term “pluripotent stem cell”, as used herein, encompasses “induced pluripotent stem cell”, or “iPSC”, a type of pluripotent stem cell derived from a differentiated non-pluripotent cell. Methods for the generation of iPSC or iPS cells are well-known in the art (Zhou et al., Stem Cells 27 (11): 2667-74 (2009); Huangfu et al, Nature Biotechnol. 26 (7): 795 (2008); Woltjen et al., Nature 458 (7239): 766-770 (2009); Zhou et al., Cell Stem Cell 8:381-384 (2009).

To generate iPSC, it is preferable to deliver one or more “reprogramming factors” to non-pluripotent cells using episomal vectors (e.g. Sendai virus, adenovirus). Unlike lentiviral and retroviral vectors, episomal vectors do not integrate into the host's cell genome and will be lost, which results in iPSC with “zero footprint”. This is preferable as no permanent viral genetic material will be present in the final iPSC.

As is appreciated by those of skill in the art, iPSC can be generated using a number of reprogramming factors, such as OCT4, KLF4, SOX2, c-MYC, NANOG, LIN28. These reprogramming factor genes are provided on episomal vectors such as are known in the art and commercially available. For example, CTS™ CytoTune™-iPS 2.1 Sendai Reprogramming Kit (Thermo Fisher Scientific, catalog number A34546) or Episomal iPSC Reprogramming Vectors (Thermo Fisher Scientific, catalog number A14703).

In addition, zero footprint human iPS cell lines are also commercially available, e.g. Human Episomal iPSC Line (Thermo Fisher Scientific, catalog number A18945)

As is known in the art, a source of non-pluripotent cells for iPSC generation could be bone marrow and blood cells, fibroblasts, keratinocytes, epithelial cells of kidney and bladder etc.

The term “dermal papilla stem cell”, as used herein, encompasses stem cells that are isolated from the hair follicle dermal compartments, such as dermal papilla and dermal sheath. As will be appreciated by those in the art, the dermal papilla and dermal sheath contain cells that are capable to induce new hair growth when transplanted alone or in combination with skin epithelial cells (Jahoda et al, Nature. 311(5986):560-2 (1984); Jahoda et al, Exp Dermatol. 10(4):229-37 (2001); McElwee et al, J Invest Dermatol. 121(6):1267-75 (2003). However, none of the current approaches for using dermal papilla stem cell were able to produce hair follicles with clinically and cosmetically satisfying results.

The terms “dermal papilla”, “dermal papilla stem cells” and “dermal sheath cup cells’ are used indistinguishably form each other and the use of one includes the use of the other.

Methods for the isolation of dermal papilla stem cell from autologous and allogeneic hair follicles are well-known in the art (Gledhill et al, Methods Mol Biol. 989:285-92 (2013); Topouzi et al. Exp Dermatol. 26(6):491-496 (2017).

As is appreciated by those of skill in the art, dermal papilla cells can also be generated by in vitro differentiation of iPSC (Gnedeva et al, PLoS One. 10(1):e0116892 (2015); Veraitch et al, Sci Rep. 7:42777 (2017).

In addition, dermal papilla primary cells and cell lines are commercially available, e. g. Human Follicle Dermal Papilla Cells (HFDPC) (PromoCell, catalogue number C-12071).

Differentiation of stem cells can be achieved through the use of culture conditions with defined combinations of nutrients, co-factors, growth factors and or small molecules. Differentiation protocols can rely on external signals or intrinsic signals, and the differentiation propensity of adult cell types varies from one cell type to another and is dependent on its hierarchy within the pluripotency level and linage commitment level.

Differentiation of stem cells towards tissue specific cells types is widely and efficiently applicable, the derivation of specific cell types can be biased by means of autocrine or paracrine signals. Paracrine signals of adjacent differentiated cells act on stem cells as lineage specific attractors, cellular identity attractors and mimicking developmental organizing centres.

A variety of differentiation protocols to derive some components of vertebrata hair follicle in vivo have been proposed (Blanpain et al., Cell 118, 635-648 (2004); Patent application CN107164310A). Similarly, there are methods for the in vitro differentiation of hair follicles from single populations of mouse pluripotent stem cells (Lee et al., Cell 22, 242-254 (2017)). However, none of them have successfully developed in vitro human hair follicles from two populations of cells, such as human pluripotent stem cells and an attractor population of human dermal papilla stem cells. Furthermore, the current protocols rely on using cell culture media supplemented with animal-derived xenogeneic growth factors (e.g. fetal bovine serum), rendering the bioengineered hair follicles unsuitable for transplantation to humans. In addition, none of the existing protocols have successfully developed transplantable bioengineered sheets containing human hair follicles.

A variety of methods for tissue fabrication for multiple tissue types have been proposed (Patent application US20170130192A1; U.S. Pat. No. 6,096,347A; Patent JP5893786B2; Patent application WO2017077985A1). However, none of them successfully developed in vitro hair follicles, or tissue sheets containing hair follicles, or integrate biodegradable material scaffolds for aiding the generation of asymmetry in the structure of said tissue sheets or in the shafts of said hair follicles.

The lack of bioengineered hair follicles and hair follicle-containing sheets with defined position of hair shafts, results in the use of natural sources for cosmetic surgeries, such us autologously donated human hair samples. However, the transplantation of autologous remaining hair follicles from a non-balding area of the scalp to a balding area possesses several limitations such as limited number of available donor hair follicles, reduced hair density and scarring at the donor area. On the other hand, using autogenous stem cells to derive the bioengineered hair follicles provides an unlimited source for autologous transplantation, which eliminates the aforementioned risks.

However, the ultimate goal of hair regenerative therapy is using allogeneic hair follicles and hair follicle-containing sheets for hair restoration. Compared to autologous cells, allogeneic pluripotent stem cells and dermal papilla stem cells are easier from a manufacturing standpoint and allow the generation of well-screened, standardized, high-quality “off-the-shelf” allogeneic hair follicles. However, due to cells' antigenicity, allogeneic hair follicles would elucidate a strong recipient's immune response and would be rejected. To circumvent the problem of rejection, reduction or elimination of cells' antigencity and generation of hypoimmunogenic universally-acceptable hair follicles will be required. Currently, there are no methods for creating hypoimmunogenic dermal papilla stem cells and hypoimmunogenic universally-acceptable hair follicles.

By “hypoimmunogenic pluripotent stem cell”, “hypoimmunogenic dermal papilla stem cell” and “hypoimmunogenic hair follicle” herein are meant a pluripotent stem cell, dermal papilla stem cell and hair follicle that do not induce an immunological response sufficient enough for allogeneic transplant rejection.

As will be appreciated by those in the art, there were several strategies proposed for generation of non-immunogenic iPSC and iPSC-derived differentiated cells (Xia et al, PNAS 116 (21) 10441-10446 (2019); Deuse et al, Nature Biotechnology 37, pages 252-258 (2019); patent application WO2018132783A1, Patent application WO2016183041A3). However, none of them successfully developed in vitro bioengineered hypoimmunogenic hair follicles, or hypoimmunogenic tissue sheets containing hair follicles,

The present application describes a method for deriving hair follicles and sheets containing hair follicles using autologous sources, and can be implemented in good manufacturing procedures pipelines and xeno-free conditions. Furthermore, using as source biodegradable grids containing and carrying said hair follicles, facilitates the downstream surgical process by augmenting the hair follicle count used and handled per surgical step.

The present invention also describes a method for deriving hair follicles and sheets containing hair follicles using allogeneic sources that are genetically-modified to avoid host immune response. This invention provides genetically-modified induced pluripotent stem cells (iPSC)_and dermal papilla stem cells (DPSC) that lack major immune antigens and produce signals preventing immune attack. Therefore, bioengineered allogeneic hair follicles formed from those iPSC and DPSC do not trigger immune responses and avoid immune rejection, which allows them to be used as universal donor hair follicles for allogeneic transplantation.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better clarify the disclosure and does not pose a limitation on the scope of the disclosure otherwise claimed. No language in the specification should be construed as indicating any non-claimed element essential to the practice of the disclosure.

The words “grid”, “mesh”, “scaffold” and “frame” are used indistinguishably form each other and the use of one includes the use of the others. In addition, the words “cavities”, “chambers” and “pockets” are used indistinguishably form each other and the use of one includes the use of the others.

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with techniques of biochemistry, enzymology, molecular and cellular biology, microbiology, genetics and protein and nucleic acid chemistry and hybridization described herein are those well-known and commonly used in the art.

The methods and techniques of the present disclosure are generally performed according to conventional methods well-known in the art and as described in various general and more specific references that are cited and discussed throughout the present specification unless otherwise indicated. See, e. g., Sambrook et al., Molecular Cloning: A Laboratory Manual, 3rd ed., Cold Spring Harbor Laboratory Press, Cold Spring Harbor, N. Y. (2001); Ausubel et al., Current Protocols in Molecular Biology, J, Greene Publishing Associates (1992, and Supplements to 2002); Handbook of Biochemistry: Section A Proteins, Vol 11976 CRC Press; Handbook of Biochemistry: Section A Proteins, Vol II 1976 CRC Press. The nomenclatures used in connection with, and the laboratory procedures and techniques of, molecular and cellular biology, protein biochemistry, enzymology and medicinal and pharmaceutical chemistry described herein are those well-known and commonly used in the art.

A cell when used herein may preferably be a mammalian cell. A mammalian cell may preferably be a cell from an human, antelope, antilopini, beaver, buffalo, caracal, cat, cheetah, chinchilla, cow, deer, eland, elephant, ermine, faux, fisher, fox, genet, giraffe, goat, golden jackal, hedgehog, horse, leopard, lynx, lion, marten, mink, monkey, ape, nutria, otter, rabbit, rhinoceros, sable, serval, sheep, shrew, stoat, swine, wolf, australian brushtail possum, mouse, rat, Camelidae, their subspecies and Pantholopinae such as: Dromedary Camel (Camelus dromedarius), Bactrian Camel (Camelus bactrianus) and it's wildtype, Llama (Llama glama), Alpaca (Vicugna pacos), Vicuña (Vicugna vicugna), Guanaco (Lama guanicoe) and Tibetan Antilope (Pantholops hodgsonii) with human preferred. A preferred mammalian cell is a stem cell or induced pluripotent stem cell or dermal papilla stem cell. The mammalian cells may be obtained from a biopsy. The mammalian cell may be from a cell line, e.g. a deposited cell line or a commonly available cell line.

### OBJECT OF THE DISCLOSURE

Some of the objects of the present disclosure are as listed herein below.

It is an object of the present disclosure to provide for methods to derive hair follicles as a source for plastic surgery.

It is an object of the present disclosure to provide for methods to derive bioengineered hair follicles containing sheets as a source for plastic surgery.

It is an object of the present disclosure to provide for methods to derive bioengineered hair follicles containing sheet with the exclusive disposition of hair filaments towards one side or facet of said bioengineered hair follicles containing sheet.

It is another object of the present disclosure to provide for a method to derive bioengineered hair follicles avoiding the need for allogeneic hair follicle donation.

It is another object of the present disclosure to provide for autologous bioengineered hair follicles to avoid the risk for tissue immune rejection.

It is yet another object of the present disclosure to provide bioengineered hypoimmunogenic hair follicles from universal donor dermal papilla stem cells and induced pluripotent stem cells.

### Items

A dermal papilla stem cell derived from a mammal, wherein said mammal cell is selected from human, antelope, antilopini, beaver, buffalo, caracal, cat, cheetah, chinchilla, cow, deer, eland, elephant, ermine, faux, fisher, fox, genet, giraffe, goat, golden jackal, hedgehog, horse, leopard, lynx, lion, marten, mink, monkey, ape, nutria, otter, rabbit, rhinoceros, sable, serval, sheep, shrew, stoat, swine, wolf, australian brushtail possum, mouse, rat, Camelidae, their subspecies and Pantholopinae such as: Dromedary Camel (Camelus dromedarius), Bactrian Camel (Camelus bactrianus) and it's wildtype, Llama (Llama glama), Alpaca (Vicugna pacos), Vicuña (Vicugna vicugna), Guanaco (Lama guanicoe) and Tibetan Antilope (Pantholops hodgsonii) with human preferred.

The mammal dermal papilla stem cell of item 1, wherein said mammal cell markers is selected from Sox2-positive, Sox9-positive, Nestin-positive, P63-positive, CD133-positive, AP-positive, alpha-SMA-positive or combinations of the above.

A pluripotent stem cell derived from a mammal, wherein said mammal cell is selected from human, antelope, antilopini, beaver, buffalo, caracal, cat, cheetah, chinchilla, cow, deer, eland, elephant, ermine, faux, fisher, fox, genet, giraffe, goat, golden jackal, hedgehog, horse, leopard, lynx, lion, marten, mink, monkey, ape, nutria, otter, rabbit, rhinoceros, sable, serval, sheep, shrew, stoat, swine, wolf, australian brushtail possum, mouse, rat, Camelidae, their subspecies and Pantholopinae such as: Dromedary Camel (Camelus dromedarius), Bactrian Camel (Camelus bactrianus) and it's wildtype, Llama (Llama glama), Alpaca (Vicugna pacos), Vicuña (Vicugna vicugna), Guanaco (Lama guanicoe) and Tibetan Antilope (Pantholops hodgsonii) with human preferred.

The mammal pluripotent stem cell of item 3, wherein said pluripotent stem cell type is selected from embryonic stem cell, primed stem cell or induced pluripotent stem cell, with induced pluripotent stem cell preferred.

A nucleic acid molecule, wherein said nucleic acid molecule comprises at least one nucleotide sequence encoding at least one fitness gene, selection marker indicating homologous or heterologous recombination when integrated in the genome of a vertebrata cell, wherein the selection markers when being expressed confers chemical resistance or is optically discriminable, e.g. in FACS or any fluorescence guided capture, and wherein the nucleotide sequence encoding a selection marker indicating homologous or heterologous recombination in a vertebrata cell is flanked 5′ and or 3′ by nucleotide sequences that are homologous to nucleotide sequences of a nucleic acid sequence present in the vertebrata cell.

The nucleic acid molecule of item 5, wherein said nucleic acid molecule comprises a chemical resistance selection marker selected from neomycin resistance, hygromycin resistance, HPRT1, puromycin resistance, puromycin N-acetyl-transferase, blasticidin resistance, G418 resistance, phleomycin resistance, nourseothricin resistance or chloramphenicol resistance.

The nucleic acid molecule of item 5, wherein said optical discriminability is different emission wavelength. The nucleic acid molecule of item 5, wherein said selection marker indicating homologous or heterologous recombination is a fluorescent protein.

The nucleic acid molecule of item 7, wherein said fluorescent protein is selected from Sirius, SBFP2, Azurite, EBFP2, mKalamal, mTagBFP2, Aquamarine, ECFP, Cerulean, mCerulean3, SCFP3A, mTurquoise2, CyPet, AmCyanl, mTFP1, MiCy, iLOV, AcGFPI, sfGFP, mEmerald, EGFP, mAzamiGreen, cfSGFP2, ZsGreen, mWasabi, SGFP2, Clover, mClover2, EYFP, mTopaz, mVenus, SYFP2, mCitrine, YPet, ZsYellowl, mPapayal, mKO, mOrange, mOrange2, mK02, TurboRFP, mRuby2, eqFP611, DsRed2, mApple, mStrawberry, FusionRed, mRFP1, mCherry, mCherry2, dTOMATO, tdTOMATO, tagBFP, photoactivatable or photoswitchable fluorescent protein.

The nucleic acid molecule of item 5, wherein said fitness gene is selected from telomerase, Ras, Abl, Akap13, Araf, Tim, Atf, Axl, Bcl, Braf, Brca, Brip, Cbl, Csf1r, Dapk, Dek, Dusp, Egf, Egfr, Erbb, Erg, Ets, Ewsr, Fes, Fgf, Fgfr, Flcn, Fos, Frap, Fus, Hras, Gli, Gpc, Neu, Hgf, Irf, Junb, Kit, Kras, Lck, Lco, Mapk, Mcf, Mdm2, Met, Mlh, Mmd, Mos, Mras, Msh, Myb, Myc, Lmyc, Nmyc, Ele1, Nf1, Trk, Can, Ovc, Tp53, Palb2, Pax3, Pdgfb, Pim, Pml, Pms, Wip, Pten, Pvt, Raf, Craf, Rb, Rras, Mcf, Smad, Smurf, Src, Stat, Tdgf, Tgfbr, Erba, Tgf, Tif, Tnc, Trk, Tusc, Usp, Wnt, Wt, Vhl, or combinations of the above.

The nucleic acid molecule of items 9, wherein said nucleotide sequence encoding a fitness gene comprises a promoter driving expression of said selection markers.

The nucleic acid molecule of item 10, wherein said promoter is constitutive or inducible.

The nucleic acid molecule of any one of the preceding items, wherein homologous or heterologous recombination is induced by random integration, TALENs, ZFNs, meganucleases, or CRISPR nuclease.

The vertebrata dermal papilla stem cell of item 1, wherein said dermal papilla stem cell is engineered to contain at least one nucleic acid molecule of item 5, at least one chemical selection marker of item 6 and/or at least one optically active selection marker of item 7 and 8, at least one fitness gene of item 9.

The vertebrata pluripotent stem cell of item 3, wherein said pluripotent stem cell is engineered to contain at least one nucleic acid molecule of item 5, at least one chemical selection marker of item 6 and/or at least one optically active selection marker of item 7 and 8, at least one fitness gene of item 9.

A grid of blended biodegradable substrates, wherein said substrates blend components is selected from polyglycolic acid, polyacetic acid, epsilon-caprolactones, polydioxanones, lactides, hydrogels, or combinations of the above.

A extracellular matrix blend, wherein said matrix blend components is selected from laminin, fibronectin, collagen, heparan sulfate, chondroitin sulfate, keratan sulfate, hyaluronic acid, elastin, integrin, cadherin, selectin, connexins, claudins, occludins, and chemically modified extracellular matrix proteins, or combinations of the above.

Three-dimensional cell aggregate composed of cellular mixtures of item 1 and item 3, and extracellular matrix item 16.

A media blend to facilitate cell aggregate formation, wherein said media components is selected from Calcium chloride, Ferric nitrate, Magnesium sulfate, Potassium chloride, Sodium bicarbonate, Sodium chloride, Sodium phosphate monobasic, L-arginine, L-cystine, glycine, L-histidine, L-isoleucine, L-leucine, L-lysine, L-methionine, L-phenylalanine, L-serine, L-threonine, L-tryptophan, L-tyrosine, L-valine, Choline chloride, Folic Acid, myo-Inositol, Niacinamide, D-Pantothenic acid, Pyridoxal, Pyridoxine, Riboflavin, Thiamine, D-Glucose, Pyruvic acid, L-Glutamine, L-proline, L-hydroxyproline, reduced glutathione, ascorbic acid, Iron saturated transferrin, Insulin, albumin, L-alanine, L-asparagine, L-aspartate, L-glutamate, beta-mercaptoethanol, L-alanyl-glutamine, Y-27632, Rho-associated coiled-coil containing protein kinase inhibitor, or combinations of the above.

A media blend to facilitate the patterning and paracrine signalling between cells item 1 and item 3, wherein said media components is selected from Calcium chloride, Cupric sulfate, Ferrous sulfate, Magnesium chloride, Potassium chloride, Sodium bicarbonate, Sodium chloride, Sodium phosphate dibasic, Zinc sulfate, L-alanine, L-asparagine, L-arginine, L-aspartic acid, L-cystine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, L-valine, D-biotin, Choline chloride, Folic acid, myo-Inositol, Niacinamide, D-Pantothenic acid, Pyridoxine, Riboflavin, Thiamine, Vitamin B12, D-Glucose, Hypoxanthine, Linoleic acid, Putrescine, Pyruvic acid, Thioctic acid, Thymidine, Sodium bicarbonate, Magnesium sulfate, Potassium Nitrate, Sodium phosphate monobasic, Sodium selenite, HEPES, Arachidonic acid, Cholesterol, DL-alpha-tocopherol acetate, Ethyl alcohol, Linoleic acid, Linolenic acid, Myristic acid, Oleic acid, Palmitic acid, Palmitoleic acid, Polyoxyethylene-polyoxypropylene copolymer, Stearic acid, Tween, albumin, Insulin, Transferrin, 1-thioglycerol, FGF2, BMP4, 4-(5-Benzol[1,3]dioxol-5-yl-4-pyridin-2-yl-1H-imidazol-2-yl)-benzamide, 4-[6-[4-(1-Piperazinyl)phenyl]pyrazolo[1,5-a]pyrimidin-3-yl]-quinoline, dorsomorphin, L-alanyl-glutamine, item 6, or combinations of the above.

A media blend to facilitate the maturation of cellular aggregates item 7, patterning and paracrine signalling between cells item 1 and item 3, and maturation of cellular aggregates item 7 into hair follicles, wherein said media components is selected from Ammonium molybdate, Ammonium metavandate, Cupric sulfate, Ferrous sulfate, Manganese sulfate, Magnesium sulfate, Nickel chloride, Sodium metasilicate, Sodium selenite, Sodium phosphate dibasic, Stannous chloride, L-aspartic acid, L-glutamic acid, L-glutamine, Calcium chloride, Ferric nitrate, Magnesium chloride, Potassium chloride, Sodium bicarbonate, Sodium chloride, Sodium phosphate monobasic, Zinc sulfate, L-alanine, L-asparagine, L-arginine, L-cystine, glycine, L-histidine, L-isoleucine, L-leucine, L-lysine, L-methionine, L-phenylalanine, L-proline, L-serine, L-threonine, L-tryptophan, L-tyrosine, L-valine, Choline chloride, Folic acid, Niacinamide, D-Pantothenic acid, Pyridoxal, Riboflavin, Thiamine, Vitamin B12, inositol, D-Glucose, HEPES, Pyruvic acid, D-biotin, myo-Inositol, Hypoxanthine, Linoleic acid, Putrescine, DL-Thioctic Acid, Thymidine, Transferrin, Insulin, Progesterone, Catalase, reduced glutathione, Superoxide dismutase, T3, L-carnitine, Ethanolamine, D+-galactose, Corticosterone, Linolenic acid, DL alpha tocopherol, DL alpha tocopherol acetate, Oleic acid, Pipecolic acid, albumin, L-alanyl-glutamine, beta-mercaptoethanol, FGF2, BMP4, 4-(5-Benzol[1,3]dioxol-5-yl-4-pyridin-2-yl-1H-imidazol-2-yl)-benzamide, 4-[6-[4-(1-Piperazinyl)phenyl]pyrazolo[1,5-a]pyrimidin-3-yl]-quinoline, dorsomorphin, L-alanyl-glutamine, item 6, or combinations of the above.

A polymer surface, wherein said surface is selected from poly-acrylates, poly-ornithine, poly-olefines, hydrogels, poly-amino acids, and poly-peptides.

A bioengineered hair follicle, wherein said bioengineered hair follicle is composed of the derivatives of cells item 1 and item 3, and extracellular matrix item 16, cultured successively on media item 18, item 19, and item 20, and produced from matured derivatives of cell aggregate item 17.

A loaded biodegradable grid, wherein said loaded biodegradable grid is composed of the biodegradable grid item 15 containing at least one cellular aggregate item 17.

A bioengineered hair follicles containing sheet, wherein said bioengineered hair follicles containing sheet is composed of the loaded biodegradable grid item 23, cultured successively on media item 18, item 19, and item 20, and wherein successive culture on media item 18, item 19 and item 20 results in the conversion of cellular aggregates item 17 into hair follicles item 22.

Hypoimmunogenic dermal papilla stem cell of item 1, wherein said hypoimmunogenic cell comprises of one or more of the following: reduced expression and/or deletion one or more genes of Major Histocompatibility Antigen Class I (HLA-I) including HLA-A, HLA-B, HLA-C; reduced expression and/or deletion one or more genes of Major Histocompatibility Antigen Class II (HLA-II) including HLA-DP, HLA-DR, HLA-DQ; reduced expression and/or deletion of B2M, CIITA, NLRC5 and combinations thereof; increased expression or activity of CD47, PD-L1, CTLA4-Ig, IL-35, HLA-E, HLA-G, C1-inhibitor and combinations thereof.

Hypoimmunogenic cell of item 25, wherein said hypoimmunogenic cell is created by using genome editing technologies including CRISPR/Cas, TALEN, CRISPR/Cas, Zinc finger (ZNF), viral, plasmid, interfering RNA, with CRISPR/Cas preferred.

Hypoimmunogenic pluripotent stem cell of item 3, wherein said hypoimmunogenic cell comprises of one or more of the following: reduced expression and/or deletion one or more genes of Major Histocompatibility Antigen Class I (HLA-I) including HLA-A, HLA-B, HLA-C; reduced expression and/or deletion one or more genes of Major Histocompatibility Antigen Class II (HLA-II) including HLA-DP, HLA-DR, HLA-DQ; reduced expression and/or deletion of B2M, CIITA, NLRC5 and combinations thereof; increased expression or activity of CD47, PD-L1, CTLA4-Ig, IL-35, HLA-E, HLA-G, C1-inhibitor and combinations thereof.

Hypoimmunogenic pluripotent stem cell of item 27, wherein said hypoimmunogenic cell is created by using genome editing technologies including CRISPR/Cas, TALEN, CRISPR/Cas, Zinc finger (ZNF), viral, plasmid, interfering RNA, with CRISPR/Cas preferred.

An in vitro method for manufacturing and deriving bioengineered hair follicles item 22 and bioengineered hair follicles containing sheet item 24:

(a) Subjecting population of dermal papillae stem cell of item 1 to liquid merging with stem cell item 3 on media item 18, and aggregate them and bring together through centrifugal force, and culture them between one hour and two days. The ratio of cells of item 1 and item 3 is adjusted within 1:1 to 1:1×10e5. The combined count of cells item 1 and item 3 per cellular aggregate ranges between 100 and 1×10e5. Cell populations are alternatively aggregated inside the spaces of biodegradable grid item 15.
(b) Coating of the three-dimensional aggregate in (a) and liquid coat it with extracellular matrix item 16 to generate the cellular aggregate item 17. Cell aggregates are alternatively coated inside the spaces of biodegradable grid item 15 yielding item 23.
(c) Subjecting the cellular aggregate item 17 to culture on media item 19 between one hour and eight days. Cell aggregates item 17 are alternatively deposited into the spaces of biodegradable grid item 15 yielding item 23.
(d) Subjecting the cellular aggregate item 17 in (c), or item 23 in (c), and patterned with media item 19 to media item 20 between seven and eighty days.
(e) Item 17 in (d) results in a bioengineered hair follicle item 22, and item 23 in (d) results in a bioengineered hair follicle containing sheet item 24.
(f) Collection of the resulting suspension hair follicles item 22 in (e), or resulting biodegradable hair follicles containing sheet item 24 in (e).

A method for deriving autologous bioengineered hair follicles or autologous bioengineered hair follicles containing sheets.

(g) Obtain dermal papillae stem cells item 1 from target individual, and obtain induced pluripotent stem cells item 3 from target individual.
(h) Derive bioengineered hair follicles or bioengineered hair follicles containing sheets from target individual cells in (g), using method item 29.

A method for deriving allogeneic hypoimmunogeneic bioengineered hair follicles or allogenic hypoimmunogeneic bioengineered hair follicles containing sheets.
(i) Obtain dermal papillae stem cells item 25 from a donor or commercial supplier, and obtain induced pluripotent stem cells item 27 from a donor or commercial supplier.
(j) Derive bioengineered hair follicles or bioengineered hair follicles containing sheets from cells in (i), using method item 29

One or more cavities integrated and contained in the biodegradable grid item 15, wherein said cavities includes a wide opening towards one side of the grid and a narrow opening towards the opposite side of the grid.

### BRIEF DESCRIPTION OF DRAWINGS

The accompanying drawings are included to provide a further understanding of the present disclosure and are incorporated in and constitute a part of this specification. The drawings illustrate exemplary embodiments of the present disclosure and, together with the description, serve to explain the principles of the present disclosure.

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates a proposed biodegradable grid scaffold item 15 and biodegradable hair follicles containing sheet item 24 in accordance with an exemplary embodiment of the present disclosure.

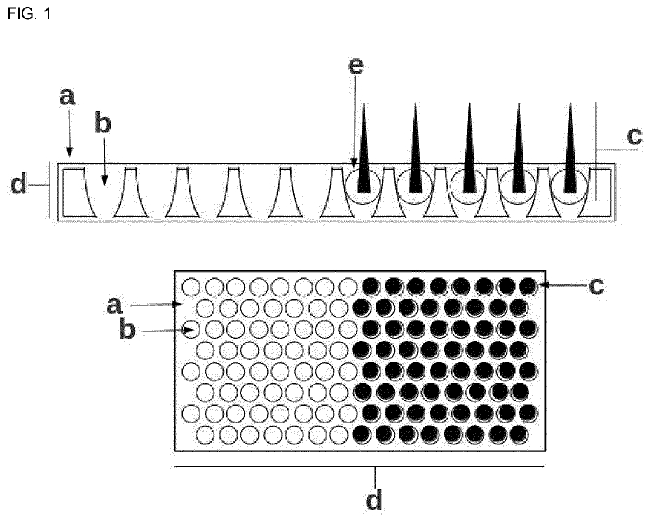

a—item 15 grid of biodegradable substrates blend
b—item 27 cavities included in item 15
c—item 22 bioengineered hair follicle
d—item 24 bioengineered hair follicles containing sheet
e—mature cell aggregate item 17

FIG. 2 illustrates a proposed three-dimensional cell aggregate item 17 and bioengineered hair follicle item 22 in accordance with an exemplary embodiment of the present disclosure.

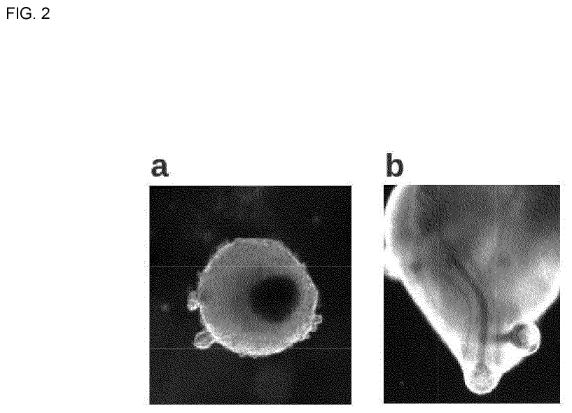

a—item 17 three dimensional cell aggregate
b—item 22 bioengineered hair follicle

FIG. 3 illustrates a proposed culture condition timeline with media blend sequences of item 18, item 19 and item 20. Also shows the manufacturing steps of loaded biodegradable grid item 23, and bioengineered hair follicles containing sheet item 24 in accordance with an exemplary embodiment of the present disclosure.

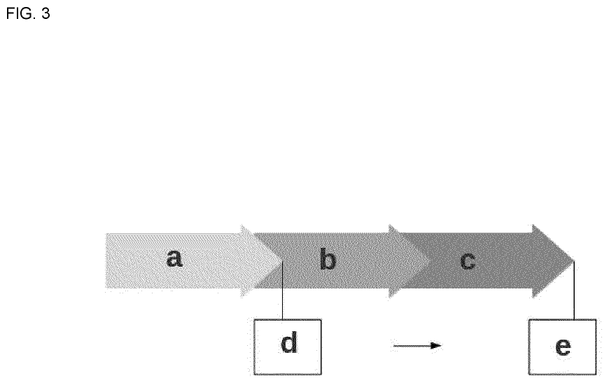

a—item 18 culture media blend
b—item 19 culture media blend
c—item 20 culture media blend
d—item 23 loaded biodegradable grid item 15 containing cell aggregate item 17
e—item 24 bioengineered hair follicles containing sheet, containing bioengineered hair follicles item 22

FIG. 4 illustrates immunological responses that activate and inhibit allogenic transplant rejection

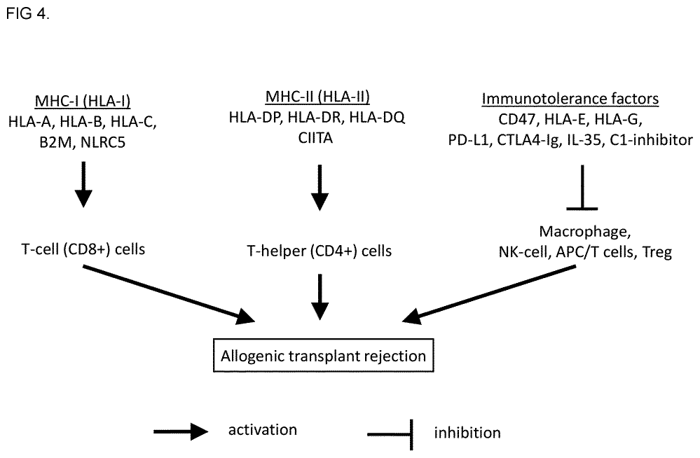

FIG. 5 illustrates a proposed hypoimmunogenic hair follicle-producing three-dimensional cell aggregate item 17 in accordance with an exemplary embodiment of the present disclosure.

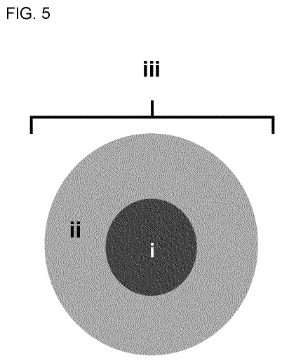

i—dermal papilla stem cell item 25
ii—pluripotent stem cell item 27
iii—three dimensional cell aggregate item 17 composed of cellular mixtures of item 25 and item 27, and extracellular matrix item 16

FIG. 6 Experimental timeline of de novo hair derivation. Staring with from iPSC and DPSC cultures EB-assembly was performed in U-Bottom ULA plates. After formation the EBs were subjected to CDM for a two-step patterning process.

FIG. 7 Day 5 (30% iPSC)—DPSC go to the center, like previously, indicated by the GFP negative area of the EB (arrow)

FIG. 8 Day 35 (20180502): Cond. 1, 90% iPSC, 6

FIG. 9 Day 74 (20180610), organoid No. 16.

Bottom: GFP-positive (no direct involvement of DPSC)

FIG. 10 Day 78 (20180614), 90%, Cond. 1, Organoid No. 13

FIG. 11. Day 82, Cond. 1, 50%, No. 16

FIG. 12 Day 82, 90%, Cond. 1, No. 13

FIG. 13 Day 82, 90%, Cond. 2, No. 13

FIG. 14 Day 92, 90%, Cond. 1, No. 13

FIG. 15 Day 92, 95%, Cond. 3, No. 32

Middle: GFP; Bottom: merge (Fixation o/n 20180629 in 4% PFA, 1×PBS)

### EXPERIMENTAL SECTION

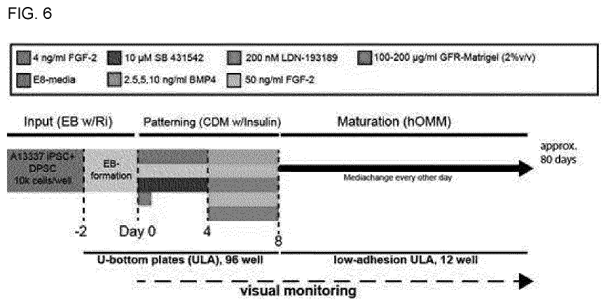

Based on the available literature Lee et al 2018 (doi: 10.1016/j.celrep.2017.12.007) the inventor developed a method to also derive human de novo hair follicle containing organoids. Lee et al 2018 was based on Koehler et al 2013 (doi: 10.1038/nature12298). Consequently, the inventor assumed that an approach based on a similar publication from Koehler 2017 (doi: 10.1038/nbt.3840) could lead to a similar success when using mature human induced pluripotent stem cells (iPSC) as a starting population. Since the inventor also had patient derived dermal papilla stem cells (DPSC) extracted from FUE-hair follicles available, the inventors assumed these cells could have a positive effect on potential de novo hair follicle assembly. In the following months, the inventor tested a variety of different combinations based on Koehler 2017 and our own protocol, to screen for potential successful combinations. Taking in to account the mouse versus human developmental timeframe, the inventor expected the de novo HF assembly to be respectively slower. Here the inventor outlines one first successful combination (FIG. 6) resulting in the first, yet characterized, organoids containing HF within a time-frame of 70-80 days of maturation. Interestingly, this approach however, was particularly successful in combinations containing DPSC. iPSC-only conditions as shown in all available papers made it barely beyond the EB-state. To be able to discriminate between the combination of iPSC and DPSC, the inventor used GFP-tagged iPSC (A13337, Gibco). During the first steps of embryoid body (EB) formation the cells were visually clearly separable, with the DPSC centered inside, which is maybe not surprising due to their mesodermal origin, and the iPSC engulfing the DPSC. Quickly the GFP+iPSC based cells overgrew and throughout the whole maturation of the organoid clearly GFP-negative cells were not observable anymore.

### Organoid Preparation and Cultivation Protocol:

(i) Have (GFP-/for DPSC mix approach) wild-type iPSC in expansion culture
(ii) When the cells are 80% confluent, aspirate the E8 medium (and wash the cells three times with PBS at RT).
(iii) Cells were dissociated with StemPro Accutase (Invitrogen, cat. no. A1110501) for ˜5 min at 37 C
(iv) Collect the dissociated cells into 1 ml of E8-Ri (ectodermal differentiation)—or desired EB forming media medium and transfer them into a 2-ml microcentrifuge tube.
(v) Break the cell clumps into single cells by pipetting with a P1000 tip. Pellet the cells by centrifugation at 200 rcf for 5 min at RT.
(vi) Completely remove the supernatant and resuspend the cell pellet in 1 ml of EB-Ri Y27632 (5 uM) medium.
(vii) Forcefully pipette 1 ml of EB-Ri medium through a cell-strainer-top test tube to prime the strainer.
(viii) Pipette the 1 ml of ES cell suspension dropwise onto the cell strainer. Next, pipette 1 ml of fresh EB-Ri medium dropwise onto the cell strainer. There should be 3 ml in the test tube.
(i) Mix the cell suspension by pipetting with a P1000 tip, and then determine the concentration of cells with a haemocytometer.
(j) Dilute the appropriate volume of cell suspension in xy ml of fresh EB-Ri medium (5 uM Ri) to acquire a final concentration of 50,000 cells per ml—to achieve 5,000 (used 10 k) cells per organoid (at least 10 ml for a complete 96er plate). Invert the tube several times to mix.
(k) Pour the cell suspension into a reservoir and aliquot 100 μl of cell suspension into each well of two 96-well plates with a multichannel pipette.
(l) Centrifuge the U-bottom plate at 200 rcf for 3 min.
   Use 1300 ul total volume
   NOTE: Here some of the extracted patient DPSC can be mixed in to mimic and induce HF growth
   Which will be a mix of GFP-iPSC (A13337, p14) and unlabelled primary DPSC (004A, p6).

| 100% iPSC | |
|---|---|
| 95% iPSC | (1225 iPSC/65 ul DPSC) |
| 90% | (1170 iPSC/130 ul DPSC) |
| 85% | (1105 iPSC/195 ul DPSC) |
| 80% | (1040 iPSC/260 ul DPSC) |
| 50% | (750 iPSC/750 ul DPSC) |
| 40% | (720 iPSC/780 ul DPSC) |
| 30% | (390 iPSC/910 ul DPSC) |

(m) Place the plates in a 37° C. incubator with 5.0% CO₂ for 24 h.
(n) After 24h, very carefully replace medium by fresh EB-medium, without Rock-inhibitor
(o) After another 24h, replace the medium by CDM containing the different combinations of patterning factors.
(p) After 4 days add 25 ul of CDM containing factors as indicated.
(q) After 4 days add 25 of fresh CDM w.o growth factors
(r) At day 12 of pre-patterning the resulting patterned EBs were moved to 12 well ULA plates and incubated in 500 ul hOMM. Plates were incubated at dynamic conditions, at 50 rpm shaking.
(s) 50% media change was performed every other day using fresh hOMM.

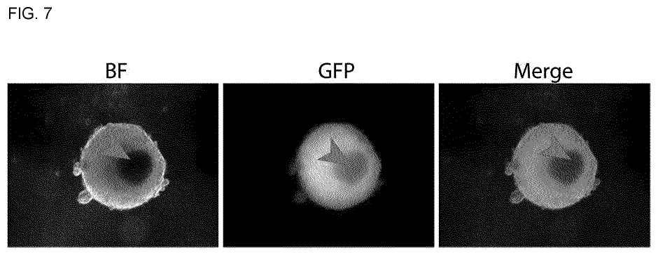

### Visual Progression in FIG. 7.

Day 8: hOMM cultivation only, media change 50% every other day (500 ul OMM volume).

Day 12 20% DPSC (DPSC not visible anymore, organoids form similar structures, theoretically too early):

Similar to previous approaches

Day 35 (20180502):

Cond. 1, 90% iPSC, 6:

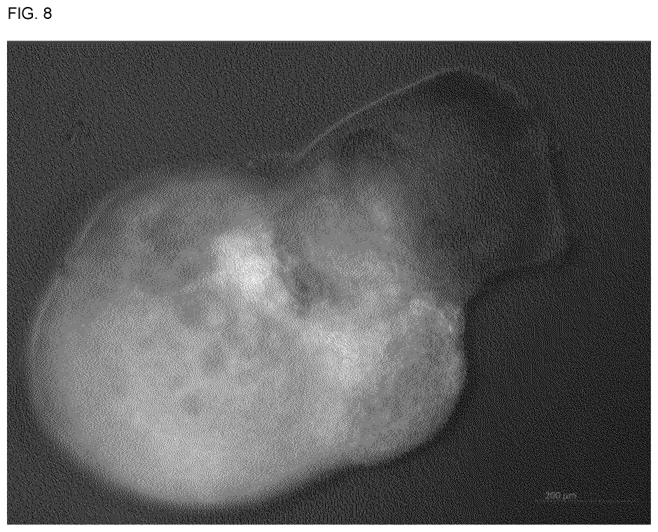

FIG. 8.

Day 74 (20180610), organoid No. 16:

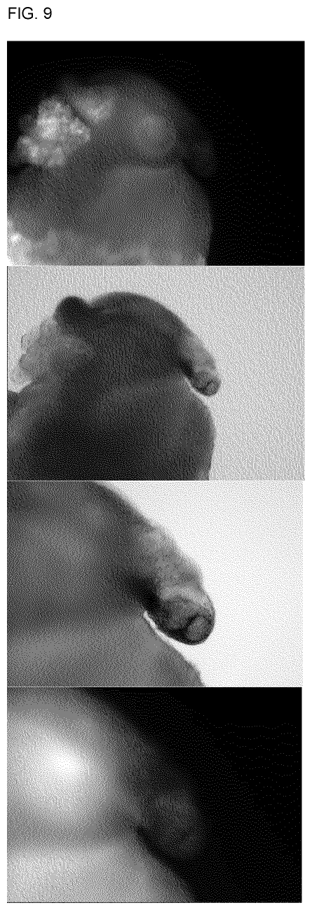

FIG. 9

Day 78 (20180614), 90%, Cond. 1, Organoid No. 13,

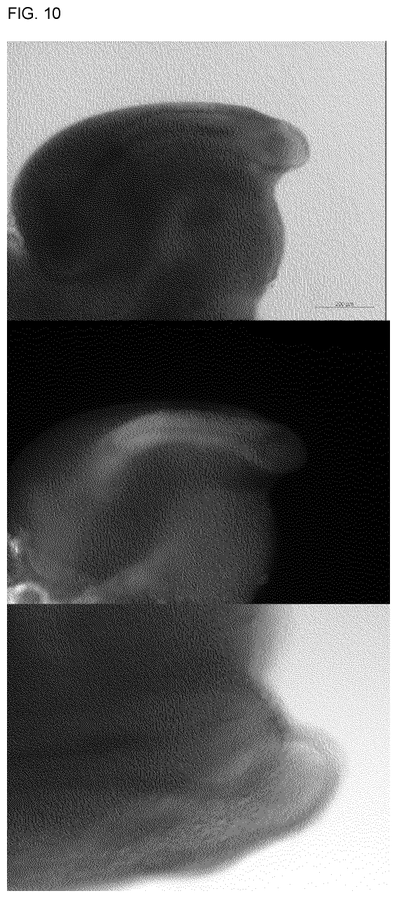

FIG. 10

Day 82, Cond. 1, 50%, No. 16

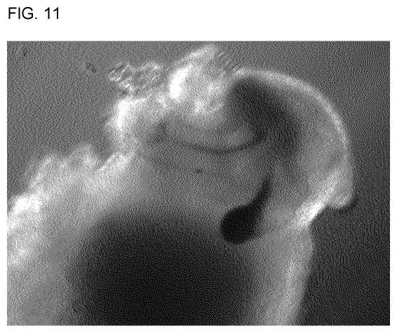

FIG. 11.

Day 82, 90%, Cond. 1, No. 13

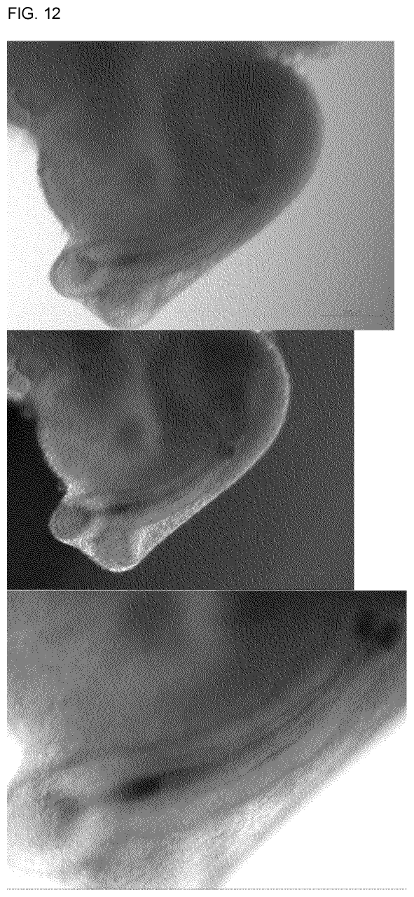

FIG. 12

Day 82, 90%, Cond. 2, No. 13

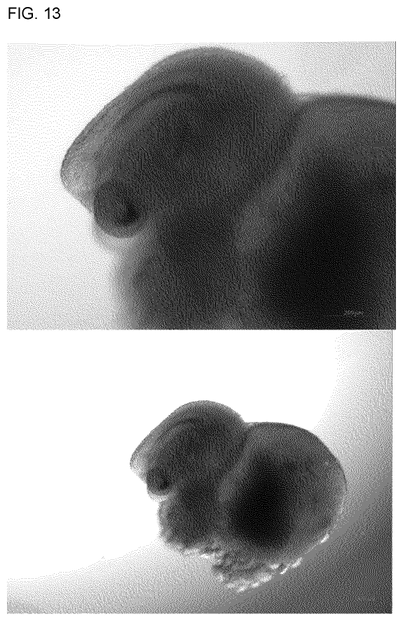

FIG. 13

Day 92, 90%, Cond. 1, No. 13

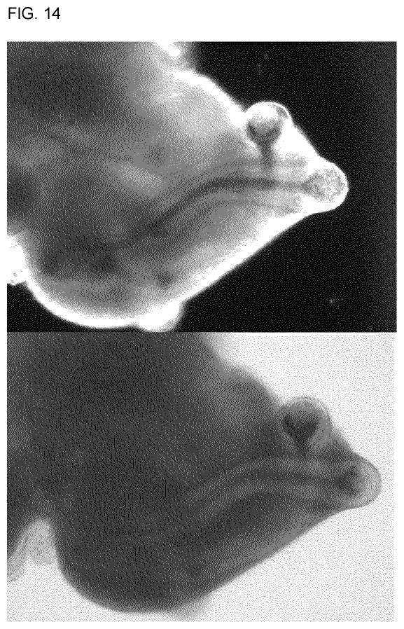

FIG. 14

Day 92, 95%, Cond. 3, No. 32

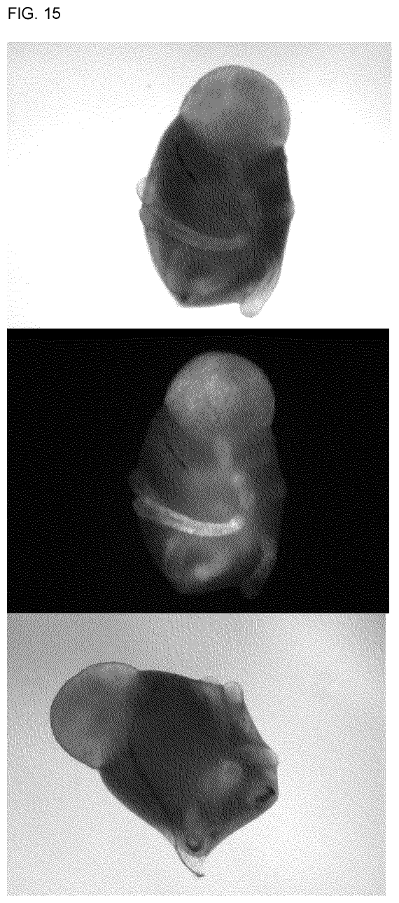

FIG. 15

### SUMMARY

It takes around 80 Days to get the first Hair follicles. Survivability in the conditions with DPSC mixed in is good, allowing the development of Hair follicles de novo in the dish in some compositions.

The protocol is flexible allowing the de novo formation in different BMP4 conditions.

### Material Overview:

### EB-Formation Medium

### EB Medium (50 mL):

40 mL KnockOut ™ DMEM (TS: 10829018)
10 mL KnockOut ™ Serum Replacement (10828010)
500 μL Normocin (Ant-nr-1)
500 μL GlutaMAX ™ Supplement (35050061)
500 μL MEM Non-Essential Amino Acids Solution (11140050)
100 μL β-mercaptoethanol (100 uM from 50 mM) (31350010)

### Chemically Defined Medium

Supplementary Table 1
Chemically-Defined Differentiation Medium (CDM)

| | | | Stock | Final | | Volume | |
|---|---|---|---|---|---|---|---|
| Component | Supplier | Cat. No. | Concentration | Concentration | | used | |
| Ham's F12 | Gibco | 31765-035 | — | 49% | (v/v) | 100 | ml |
| GlutaMax | | | | | | | |
| IMDM GlutaMax | Gibco | 31980-030 | — | 49% | (v/v) | 100 | ml |
| Chemically- | Gibco | 11905-031 | 100x | 1x | | 2 | ml |
| Defined Lipid | | | | | | | |
| BSA | Sigma | A1470 | — | 5 | mg/ml | 1 | g |
| Insulin | Sigma | I9278 | 10 mg/ml | 7 | μg/ml | 140 | μl |
| Transferrin | Sigma | T8158 | 20 mg/ml | 15 | μg/ml | 150 | μl |
| 1-thioglycerol | Sigma | M6145 | 11.5M | 450 | μM | 8 | μl |
| Normocin | Invivogen | Ant-nr-1 | 50 mg/ml | 100 | μg/ml | 400 | μl |

Note:
This formulation is for 200 mL of medium which should be used for <2 weeks. We sterile filtered the medium before adding Insulin and other factors. A variation of this medium was previously used to generate cerebellar and anterior pituitary organoids^{6,7}.

### Human Organoid Maturation Medium

| | | | Stock | | Final | | Volume | |
|---|---|---|---|---|---|---|---|---|
| Component | Supplier | Cat. No. | Concentration | | Concentration | | (50 ml) | |
| Adv DMEM/F12 | Gibco | 12491-015 | — | | 49% | (v/v) | 24.5 | ml |
| Neurobasal | Gibco | 21103-049 | — | | 49% | (v/v) | 24.5 | ml |
| N2 supplement | Gibco | 17502-048 | 100x | | 0.5x | | 250 | μl |
| B27 -Vitamin A | Gibco | 12587-010 | 50x | | 1x | | 500 | μl |
| GlutaMAX | Gibco | 35050-079 | 100x | | 1x | | 500 | μl |
| Mercaptoethanol | Gibco | 21985-015 | 55 | mM | 0.1 | mM | 91 | μl |
| Normocin | Invivogen | Ant-nr-1 | 50 | mg/ml | 100 | μg/ml | 100 | μl |

Note:
This formulation is for 50 mL of medium, which should be used for <2 weeks. This medium is a custom-made hybrid of two media previously used to generate cerebral and gastric organoids^{8,9}. B27 without Vitamin A was used to limit the influence of endogenously produced retinoic acid.

1. A bioengineered hair follicle comprising: one or more bioengineered dermal papillae stem cell-induced and pluripotent stem cell-derived hair follicles, and containing hair bulge, hair shaft, and protruding hair filament, also a cell-composed cortex containing stem cell-derived synthetic interfollicular epidermis, and intertwining extracellular matrix.

2. The dermal papillae stem cell of claim 1, wherein the cell is preferably a dermal papillae stem cell, or a dermal papillae stem cell line, and said cell is preferably genetically modified for unlimited expansion, or immortalized, or engineered to over-express extracellular matrix proteins or attachment proteins, or genetically engendered to eliminate immunogenicity and to avoid allogenic transplant rejection, or combinations of the above.

3. The extracellular matrix of claim 1, wherein the matrix is preferably composed of combinations of laminin, fibronectin, collagen, heparan sulfate, chondroitin sulfate, keratan sulfate, hyaluronic acid, elastin, integrin, cadherin, selectin, connexins, claudins, occludins, and or chemically modified extracellular matrix proteins.

4. The bioengineered hair follicle and synthetic interfollicular epidermis of claim 1,
(a) wherein the bioengineered hair follicle and synthetic interfollicular epidermis are derived from stem cells; wherein a stem cell may preferably be embryonic stem cells or induced pluripotent stem cell, with induced pluripotent stem cell being preferred; or
(b) wherein the bioengineered hair follicle and synthetic interfollicular epidermis cell is preferably a genetically modified cell, wherein the modification results in its unlimited expansion, or immortalization, or constitutive expression, or inducible expression, or permanently encoding expression, or excisable encoding expression of telomerase, Ras, Abl, Akap13, Araf, Tim, Atf, Axl, Bcl, Braf, Brca, Brip, Cbl, Csflr, Dapk, Dek, Dusp, Egf, Egfr, Erbb, Erg, Ets, Ewsr, Fes, Fgf, Fgfr, Flcn, Fos, Frap, Fus, Hras, Gli, Gpc, Neu, Hgf, Irf, Junb, Kit, Kras, Lck, Lco, Mapk, Mcf, Mdm2, Met, Mlh, Mmd, Mos, Mras, Msh, Myb, Myc, Lmyc, Nmyc, Ele1, Nf1, Trk, Can, Ovc, Tp53, Palb2, Pax3, Pdgfb, Pim, Pml, Pms, Wip, Pten, Pvt, Raf, Craf, Rb, Rras, Mcf, Smad, Smurf, Src, Stat, Tdgf, Tgfbr, Erba, Tgf, Tif, Tnc, Trk, Tusc, Usp, Wnt, Wt, Vhl, and with excisability of coding sequence preferred, or genetically engendered to eliminate immunogenicity and to avoid allogenic transplant rejection, or combinations of the above.

5. (canceled)

6. The bioengineered hair follicle of claim 1, wherein (a) the bioengineered hair follicle is derived from dermal papillae stem cells; wherein a dermal papillae stem cell may preferably be Sox2-positive, Sox9-positive, Nestin-positive, P63-positive, CD133-positive, AP-positive, alpha-SMA-positive or combinations of the above; or (b) during manufacturing are preferably integrated and inserted into a biodegradable grid sheet that forces and facilitates the asymmetric positioning of protruding hair filaments to only one side of the biodegradable grid sheet.

7. The bioengineered hair follicle-producing dermal papillae stem cell of claim 1 is preferably a genetically modified cell, wherein the modification results in its unlimited expansion, or immortalization, or constitutive or inducible, or permanently encoding or excisable encoding expression of telomerase, Ras, Abl, Akap13, Araf, Tim, Atf, Axl, Bcl, Braf, Brea, Brip, Cbl, Csflr, Dapk, Dek, Dusp, Egf, Egfr, Erbb, Erg, Ets, Ewsr, Fes, Fgf, Fgfr, Flcn, Fos, Frap, Fus, Hras, Gli, Gpc, Neu, Hgf, Irf, Junb, Kit, Kras, Lck, Lco, Mapk, Mcf, Mdm2, Met, Mlh, Mmd, Mos, Mras, Msh, Myb, Myc, Lmyc, Nmyc, Ele1, Nf1, Trk, Can, Ovc, Tp53, Palb2, Pax3, Pdgfb, Pim, Pml, Pms, Wip, Pten, Pvt, Raf, Craf, Rb, Rras, Mcf, Smad, Smurf, Src, Stat, Tdgf, Tgfbr, Erba, Tgf, Tif, Tnc, Trk, Tusc, Usp, Wnt, Wt, Vhl, or combinations of the above, and with excisability of coding sequence preferred.

8. The synthetic interfollicular epidermis of claim 1, wherein the synthetic interfollicular epidermis is composed of one or more cell types; wherein a synthetic interfollicular epidermis composed cellular cortex may preferably be composed of keratinocytes, basal cells, spinous cells, granular cells, cornified cells, or combinations of the above.

9. The dermal papillae stem cells and stem cells of claim 1 and during manufacturing are preferably homogeneously distributed within the cellular aggregate; wherein said cells are encapsulated or intertwined in extracellular matrix, wherein the extracellular matrix is preferably composed of combinations of laminin, fibronectin, collagen, heparan sulfate, chondroitin sulfate, keratan sulfate, hyaluronic acid, elastin, integrin, cadherin, selectin, connexins, claudins, occludins, and or chemically modified extracellular matrix proteins.

10. (canceled)

11. The biodegradable grid sheet of claim 6 composed of (a) biodegradable substrates blend, wherein said substrates blend components is selected from polyglycolic acid, polyacetic acid, epsilon-caprolactones, polydioxanones, lactides, hydrogels, or combinations of the above, or (b) one or more cavities that support and contain the bioengineered hair follicle comprising: one or more bioengineered dermal papillae stem cell-induced and pluripotent stem cell-derived hair follicles, and containing hair bulge, hair shaft, and protruding hair filament.

12. (canceled)

13. The cellular aggregates of claim 9, generated in a media blend, wherein said media blend components is selected from Calcium chloride, Ferric nitrate, Magnesium sulfate, Potassium chloride, Sodium bicarbonate, Sodium chloride, Sodium phosphate monobasic, L-arginine, L-cystine, glycine, L-histidine, L-isoleucine, L-leucine, L-lysine, L-methionine, L-phenylalanine, L-serine, L-threonine, L-tryptophan, L-tyrosine, L-valine, Choline chloride, Folic Acid, myo-Inositol, Niacinamide, D-Pantothenic Acid, Pyridoxal, Pyridoxine, Riboflavin, Thiamine, D-Glucose, Pyruvic Acid, L-Glutamine, L-proline, L-hydroxyproline, reduced glutathione, ascorbic acid, Iron saturated Transferrin, Insulin, albumin, L-alanine, L-asparagine, L-aspartate, L-glutamate, beta-mercaptoethanol, L-alanyl-glutamine, Y-27632, Rho-associated coiled-coil containing protein kinase inhibitor, or combinations of the above and/or during manufacturing, is generated in liquid by the combination of dermal papillae stem cells, stem cells and extracellular matrix.

14. The bioengineered hair follicle of claim 1 is preformed by and through a culture period within seven days and ninety days.

15. The bioengineered hair follicles containing sheet of claim 10 and during manufacturing, is generated by culturing the contained bioengineered hair follicles within a period of seven days and ninety days.

16. (canceled)

17. The cellular aggregates of claim 9 are matured in a media blend to facilitate the patterning and paracrine signalling between dermal papillae stem cells and stem cells, wherein said media blend components is selected from Calcium chloride, Cupric sulfate, Ferrous sulfate, Magnesium chloride, Potassium chloride, Sodium bicarbonate, Sodium chloride, Sodium phosphate dibasic, Zinc Sulfate, L-alanine, L-asparagine, L-arginine, L-aspartic acid, L-cystine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, L-valine, D-biotin, Choline chloride, Folic acid, myo-Inositol, Niacinamide, D-Pantothenic Acid, Pyridoxine, Riboflavin, Thiamine, Vitamin B12, D-Glucose, Hypoxanthine, Linoleic acid, Putrescine, Pyruvic acid, Thioctic acid, Thymidine, Sodium bicarbonate, Magnesium sulfate, Potassium nitrate, Sodium phosphate monobasic, Sodium selenite, HEPES, Arachidonic acid, Cholesterol, DL-alpha-tocopherol acetate, Ethyl alcohol, Linoleic acid, Linolenic acid, Myristic acid, Oleic acid, Palmitic acid, Palmitoleic acid, Polyoxyethylene-polyoxypropylene copolymer, Stearic acid, Tween, albumin, insulin, transferrin, 1-thioglycerol, FGF2, BMP4, 4-(5-Benzol[1,3]dioxol-5-yl-4-pyridin-2-yl-1H-imidazol-2-yl)-benzamide, 4-[6-[4-(1-Piperazinyl)phenyl]pyrazolo[1·5-a]pyrimidin-3-yl]-quinoline, dorsomorphin, L-alanyl-glutamine, extracellular matrix, or combinations of the above.

18. The bioengineered hair follicles of claim 1 are derived in a media blend to facilitate the maturation of cellular aggregates, patterning and paracrine signalling between dermal papillae stem cells and stem cells and maturation of hair follicles, wherein said media blend components is selected from Ammonium molybdate, Ammonium metavanadate, Cupric sulfate, Ferrous sulfate, Manganese sulfate, Magnesium sulfate, Nickel chloride, Sodium metasilicate, Sodium selenite, Sodium phosphate dibasic, Stannous chloride, L-aspartic acid, L-glutamic acid, L-glutamine, Calcium chloride, Ferric nitrate, Magnesium chloride, Potassium chloride, Sodium bicarbonate, Sodium chloride, Sodium phosphate monobasic, Zinc sulfate, L-alanine, L-asparagine, L-arginine, L-cystine, glycine, L-histidine, L-isoleucine, L-leucine, L-lysine, L-methionine, L-phenylalanine, L-proline, L-serine, L-threonine, L-tryptophan, L-tyrosine, L-valine, Choline chloride, Folic Acid, Niacinamide, D-Pantothenic Acid, Pyridoxal, Riboflavin, Thiamine, Vitamin B12, inositol, D-Glucose, HEPES, Pyruvic acid, D-biotin, myo-Inositol, Hypoxanthine, Linoleic acid, Putrescine, DL-Thioctic Acid, Thymidine, Transferrin, Insulin, Progesterone, Catalase, reduced glutathione, Superoxide dismutase, T3, L-carnitine, Ethanolamine, D+-galactose, Corticosterone, Linolenic acid, DL alpha tocopherol, DL alpha tocopherol acetate, Oleic acid, Pipecolic acid, albumin, L-alanyl-glutamine, beta-mercaptoethanol, FGF2, BMP4, 4-(5-Benzol[1,3]dioxol-5-yl-4-pyridin-2-yl-1H-imidazol-2-yl)-benzamide, 4-[6-[4-(1-Piperazinyl)phenyl]pyrazolo[1,5-a]pyrimidin-3-yl]-quinoline, dorsomorphin, L-alanyl-glutamine, or combinations of the above.

19. A bioengineered and biodegradable hair follicles containing sheet comprising: bioengineered hair follicles of claim 1 and/or a biodegradable grid comprising the bioengineered hair follicles of claim 1, wherein during manufacturing are preferably integrated and inserted into a biodegradable grid sheet that forces and facilitates the asymmetric positioning of protruding hair filaments to only one side of the biodegradable grid sheet.

20. The bioengineered hair follicles of claim 1, and biodegradable hair follicles containing grid sheet wherein during manufacturing are preferably integrated and inserted into a biodegradable grid sheet that forces and facilitates the asymmetric positioning of protruding hair filaments to only one side of the biodegradable grid sheet and are manufactured and produced by successively culturing them on media blends wherein
(a) said media blend components is selected from Calcium chloride, Ferric nitrate, Magnesium sulfate, Potassium chloride, Sodium bicarbonate, Sodium chloride, Sodium phosphate monobasic, L-arginine, L-cystine, glycine, L-histidine, L-isoleucine, L-leucine, L-lysine, L-methionine, L-phenylalanine, L-serine, L-threonine, L-tryptophan, L-tyrosine, L-valine, Choline chloride, Folic Acid, myo-Inositol, Niacinamide, D-Pantothenic Acid, Pyridoxal, Pyridoxine, Riboflavin, Thiamine, D-Glucose, Pyruvic Acid, L-Glutamine, L-proline, L-hydroxyproline, reduced glutathione, ascorbic acid, Iron saturated Transferrin, Insulin, albumin, L-alanine, L-asparagine, L-aspartate, L-glutamate, beta-mercaptoethanol, L-alanyl-glutamine, Y-27632, Rho-associated coiled-coil containing protein kinase inhibitor, or combinations of the above;
(b) matured in a media blend to facilitate the patterning and paracrine signalling between dermal papillae stem cells and stem cells, wherein said media blend components is selected from Calcium chloride, Cupric sulfate, Ferrous sulfate, Magnesium chloride, Potassium chloride, Sodium bicarbonate, Sodium chloride, Sodium phosphate dibasic, Zinc Sulfate, L-alanine, L-asparagine, L-arginine, L-aspartic acid, L-cystine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, L-valine, D-biotin, Choline chloride, Folic acid, myo-Inositol, Niacinamide, D-Pantothenic Acid, Pyridoxine, Riboflavin, Thiamine, Vitamin B12, D-Glucose, Hypoxanthine, Linoleic acid, Putrescine, Pyruvic acid, Thioctic acid, Thymidine, Sodium bicarbonate, Magnesium sulfate, Potassium nitrate, Sodium phosphate monobasic, Sodium selenite, HEPES, Arachidonic acid, Cholesterol, DL-alpha-tocopherol acetate, Ethyl alcohol, Linoleic acid, Linolenic acid, Myristic acid, Oleic acid, Palmitic acid, Palmitoleic acid, Polyoxvethylene-polyoxypropylene copolymer, Stearic acid, Tween, albumin, insulin, transferrin, 1-thioglycerol, FGF2, BMP4, 4-(5-Benzol[1,3]dioxol-5-yl-4-pyridin-2-yl-1H-imidazol-2-yl)-benzamide, 4-[6-[4-(1-Piperazinyl)phenyl]pyrazolo[1,5-a]pyrimidin-3-yl]-quinoline, dorsomorphin, L-alanyl-glutamine, extracellular matrix, or combinations of the above; or
(c) media blend to facilitate the maturation of cellular aggregates, patterning and paracrine signalling between dermal papillae stem cells and stem cells and maturation of hair follicles, wherein said media blend components is selected from Ammonium molybdate, Ammonium metavandate, Cupric sulfate, Ferrous sulfate, Manganese sulfate, Magnesium sulfate, Nickel chloride, Sodium metasilicate, Sodium selenite, Sodium phosphate dibasic, Stannous chloride, L-aspartic acid, L-glutamic acid, L-glutamine, Calcium chloride, Ferric nitrate, Magnesium chloride, Potassium chloride, Sodium bicarbonate, Sodium chloride, Sodium phosphate monobasic, Zinc sulfate, L-alanine, L-asparagine, L-arginine, L-cystine, glycine, L-histidine, L-isoleucine, L-leucine, L-lysine, L-methionine, L-phenylalanine, L-proline, L-serine, L-threonine, L-tryptophan, L-tyrosine, L-valine, Choline chloride, Folic Acid, Niacinamide, D-Pantothenic Acid, Pyridoxal, Riboflavin, Thiamine, Vitamin B12, inositol, D-Glucose, HEPES, Pyruvic acid, D-biotin, myo-Inositol, Hypoxanthine, Linoleic acid, Putrescine, DL-Thioctic Acid, Thymidine, Transferrin, Insulin, Progesterone, Catalase, reduced glutathione, Superoxide dismutase, T3, L-camitine, Ethanolamine, D+-galactose, Corticosterone, Linolenic acid, DL alpha tocopherol, DL alpha tocopherol acetate, Oleic acid, Pipecolic acid, albumin, L-alanyl-glutamine, beta-mercaptoethanol, FGF2, BMP4, 4-(5-Benzol[1,3]dioxol-5-yl-4-pyridin-2-yl-1H-imidazol-2-yl)-benzamide, 4-[6-[4-(1-Piperazinyl)phenyl]pyrazolo[1,5-a]pyrimidin-3-yl]-quinoline, dorsomorphin, L-alanyl-glutamine, or combinations of the above.

21. The hypoimmunogenic dermal papilla stem cells of claim 2 comprising reduced expression and/or deletion one or more genes of Major Histocompatibility Antigen Class I (HLA-I) including HLA-A, HLA-B, HLA-C; reduced expression and/or deletion one or more genes of Major Histocompatibility Antigen Class II (HLA-II) including HLA-DP, HLA-DR, HLA-DQ; reduced expression and/or deletion of B2M, CIITA, NLRC5 and combinations thereof; increased expression or activity of CD47, PD-L1, CTLA4-lg, IL-35, HLA-E, HLA-G, C1-inhibitor and combinations thereof.

22. The hypoimmunogenic bioengineered hair follicles of claim 1, or hypoimmunogenic biodegradable hair follicles containing grid sheet wherein during manufacturing are preferably integrated and inserted into a biodegradable grid sheet that forces and facilitates the asymmetric positioning of protruding hair filaments to only one side of the biodegradable grid sheet composed of the hypoimmunogenic dermal papilla stem cells comprising reduced expression and/or deletion one or more genes of Major Histocompatibility Antigen Class I (HLA-I) including HLA-A, HLA-B, HLA-C: reduced expression and/or deletion one or more genes of Major Histocompatibility Antigen Class II (HLA-II) including HLA-DP, HLA-DR, HLA-DQ; reduced expression and/or deletion of B2M, CIITA, NLRC5 and combinations thereof; increased expression or activity of CD47, PD-L1, CTLA4-lg, IL-35, HLA-E, HLA-G, C1-inhibitor and combinations thereof and/or hypoimmunogencic pluripotent stem cells selected from the group consisting of embryonic stem cells or induced pluripotent stem cells.

23. The manufacturing process of claim 20 for the generation of bioengineered hair follicles and biodegradable hair follicles containing sheet, wherein the process is implemented with;
(a) autologous embryonic stem cells or induced pluripotent stem cells, dermal papillae stem cell, or a dermal papillae stem cell line, and said cell is preferably genetically modified for unlimited expansion, or immortalized, or engineered to over-express extracellular matrix proteins or attachment proteins, or genetically engendered to eliminate immunogenicity and to avoid allogenic transplant rejection, or combinations thereof or to derive autologous bioengineered hair follicles and autologous bioengineered hair follicles containing sheets yielding non-immunogenic hair follicles supply source for cosmetic industry; or
(b) allogenic hypoimmunogenic cells comprising reduced expression and/or deletion one or more genes of Major Histocompatibility Antigen Class I (HLA-I) including HLA-A, HLA-B, HLA-C reduced expression and/or deletion one or more genes of Major Histocompatibility Antigen Class II (HLA-I I) including HLA-DP, HLA-DR, HLA-DO; reduced expression and/or deletion of B2M, CIITA, NLRC5 and combinations thereof; increased expression or activity of CD47, PD-L1, CTLA4-lg, IL-35, HLA-E, HLA-G, C1-inhibitor and combinations thereof; and/or embryonic stem cells or induced pluripotent stem cells
to derive allogenic hypoimmunogenic bioengineered hair follicles and allogenic hypoimmunogenic bioengineered hair follicles containing sheets integrated during manufacture with bioengineered hair follicle comprising: one or more bioengineered dermal papillae stem cell-induced and pluripotent stem cell-derived hair follicles, and containing hair bulge, hair shaft, and protruding hair filament, also a cell-composed cortex containing stem cell-derived synthetic interfollicular epidermis, and intertwining extracellular matrix and inserted into a biodegradable grid sheet that forces and facilitates the asymmetric positioning of protruding hair filaments to only one side of the biodegradable grid sheet yielding hypoimmunogenic hair follicles supply source for cosmetic industry.

24. (canceled)
